(19) United States
(12) Patent Application Publication
(10) Pub. No.: US 2018/0303878 A1
(43) Pub. Date: Oct. 25, 2018

(54) TREATMENT FOR INFECTION COMPOSED OF MENSTRUAL STEM CELLS

(71) Applicant: CELLS FOR CELLS, S.P.A., Santiago (CL)

(72) Inventors: Maroun KHOURY, Santiago (CL); Francisca ALCAYAGA, Santiago (CL)

(21) Appl. No.: 15/768,469

(22) PCT Filed: Oct. 14, 2016
(86) PCT No.: PCT/IB2016/056180

§ 371 Date: Apr. 13, 2018

Related U.S. Application Data
(60) Provisional application No. 62/241,564, filed on Oct. 14, 2015.

(51) Int. Cl. A61K 35/28; A61P 31/04

(52) U.S. Cl. CPC A61K 35/28; A61K 45/06; A61K 2300/00; A61P 31/04

(57) ABSTRACT

The present invention offers a solution to the lack of effective alternative treatments to fight infectious disease, preferably involving sepsis, comprising active ingredients obtained by non-intrusive and efficient methods. In particular, the present invention is the first to show that mesenchymal stem cells obtained from menstrual fluids (MenSCs) have the capacity to control infectious diseases, especially those leading to a reaction of the host body like sepsis. As shown herein, in vivo experiments illustrate that MenSCs have antibacterial activity in vitro, increase the survival rates of a mouse model for sepsis, regulate several parameters that are altered in sepsis patients and that are related with multi-organ dysfunction, such as the levels of Alanine aminotransferase (ALT), Aspartate aminotransferase (AST), alkaline phosphatase (ALP), glucose in blood, serum albumin, lung injury. Results show that in the mouse model for sepsis, MenScs also regulate the pro- and anti-inflammatory cytokine levels, reduce the loss of lymphocytes during sepsis and systemic bacterial proliferation in blood. The conditioned medium of MenSCs also increases the survival rates of mouse animals affected by sepsis. Overall, the invention offers a promising alternative method to treat infectious diseases. Since it is principally composed of stem cells present in menstrual fluid, the invention provides an ease access and repeated sampling in a non-invasive manner. Such attributes allow the rapid production of the treatment.

### FIELD OF THE INVENTION

The present invention can be included in the field of new medical treatments, wherein specific cells are used for treating a given disease or disorder. In particular, mesenchymal stem cells from the menstrual fluid are used in the present invention to treat infectious diseases.

### BACKGROUND OF THE INVENTION

An infectious disease is caused by the invasion and multiplication of microorganisms such as bacteria, viruses, and parasites that are not normally present in the body. They are transmitted from person to person by direct or indirect contact. An infection may remain localized, or it may spread through the blood or lymphatic vessels to become systemic.

Nowhere in the world of infectious diseases have yet become a negligible cause of illness and death. The number of deaths worldwide caused by pathogens and parasites was 15 million in 2010, and two-thirds of deaths were caused by around 20 species, mainly bacteria and viruses. And in 2010, in low-income countries, infectious disease was still the primary cause of death. In Western Europe, North America and other parts of the industrialized world the main cause of death is not infectious diseases anymore (Christopher Dye, After 2015: infectious diseases in a new era of health and development; Philos Trans R Soc Lond B Biol Sci. 2014). However, drug-resistant bacteria are becoming a new thread for global health worldwide. For instance, according to the world health organization (WHO), resistance of Klebsiella pneumoniae is a major cause of hospital-acquired infections such as pneumonia, bloodstream infections, and infections in newborns and intensive-care unit patients. More than half of people with an infection caused by K. pneumoniae show resistance to Carbapenem antibiotics. In some parts of the globe, one of the most widely used medicines to treat urinary tract infections, fluoroquinolone antibiotics, is now ineffective in more than half of patients due to the acquired resistance of E. Coli. Bacteria causing gonorrhea have also shown resistance to the third generation of cephalosporin in at least 10 countries (Australia, Austria, Canada, France, Japan, Norway, Slovenia, South Africa, Sweden and the United Kingdom of Great Britain and Northern Ireland). In 2014, there were about 480 000 new cases of multidrug-resistant tuberculosis, a form of tuberculosis that is resistant to the 2 most powerful anti-tuberculosis drugs. Resistance of P. falciparum malaria to the first line treatment has been confirmed in 5 countries of the Greater Mekong subregion (Cambodia, the Lao People's Democratic Republic, Myanmar, Thailand and Viet Nam). Moreover, along the Cambodia-Thailand border, P. falciparum has become resistant to almost all available antimalarial medicines, making treatment more challenging. In summary, it is becoming more and more important to find new treatments for infectious diseases that use novel strategies.

Sepsis is a specific reaction of the body to an infection insult, characterized by a hyper-immune response. It results in an intense surge of cytokines that leads to hypotension, multiple organ failure and sometimes to death. It is often accompanied by a state of relative immune paralysis caused by apoptosis of immune cells and high level of anti-inflammatory cytokines that inhibit lymphocytes and macrophages and suppress the production of pro-inflammatory cytokines. This immune paralysis is thought to cause the delayed mortality seen in some septic patients, due to their incapacity to eliminate the infection. The balance between hyper-immune response and immune paralysis varies with the patients and with the course of the illness within the same patient. Nowadays, sepsis is still a significant cause of illness and death worldwide. Indeed, it is the most frequent cause of death in non-coronary intensive care units in the occidental world, and only in the USA, each year 750 000 new cases of sepsis occur and 210 000 people die of sepsis.

Several treatments have been tried so far for sepsis. For instance, antibodies were designed to bind bacterial components that are responsible for the hyper-inflammatory response of sepsis, but the effectiveness of the treatment was not clear. Steroids also inhibit the production of anti-inflammatory cytokines and increase anti-inflammatory mediators however they often lead to death of patients caused by a secondary infection. Therapies were also designed to block cytokines such as TNFα and IL-1. However they showed limited efficacy probably due to the early and transient kinetic of these inflammatory cytokines. Immunostimulatory medicaments were shown to be effective but only in patients in the hypoimmune phase of the disease (Bernard A. M. and Bernard G. R., The immune response: targets of the treatment of severe sepsis, International Journal of Inflammation, 2012). Overall, it appears that no treatment is the perfect antidote and new treatments might be developed to cure more patients suffering sepsis.

In the last years, mesenchymal stem cells from bone marrow or adipose tissue have been reported to have antimicrobial properties (Morgan T. Sutton et al., Antimicrobial Properties of Mesenchymal Stem Cells: Therapeutic Potential for Cystic Fibrosis Infection, and Treatment; Stem Cells Int. 2016) and to reduce mortality of in vivo models of severe sepsis, (WO 2010/015929 A2; Shirley H. J. Mei S. H. J. et al., Mesenchymal Stem Cells Reduce Inflammation while Enhancing Bacterial Clearance and Improving Survival in Sepsis; Am J Respir Crit Care Med 2010). However, the isolation of Mesenchymal cells from bone marrow requires an invasive procedure that is painful for the patient and is accompanied by a risk of infection. Additionally, a very low percentage of mesenchymal cells can be isolated from a bone marrow sample. To isolate mesenchymal cells from adipose tissue, a liposuction is required which also implies some risk for the health of the donor.

Overall, more effective treatments composed by active ingredients obtained with less intrusive and more efficient procedures are required.

### BRIEF DESCRIPTION

The present invention offers a solution to the problem cited above of a lack of effective alternative treatments to fight infectious disease, preferably involving sepsis, comprising active ingredients obtained by non-intrusive and efficient methods. As shown herein, in vivo experiments illustrate that mesenchymal stem cells obtained from menstrual fluids (MenSCs) have antibacterial activity in vitro, increase survival rates of a mouse model for sepsis, regulate several parameters that are altered in sepsis patients and that are related with multiorgan dysfunction, such as the levels of Alanine aminotransferase (ALT), Aspartate aminotransferase (AST), alkaline phosphatase (ALP), glucose in blood, serum albumin, lung injury. Results show that in the mouse model for sepsis, MenScs also regulate the pro- and anti-inflammatory cytokine levels, reduce the loss of lymphocytes during sepsis and systemic bacterial proliferation in blood. The conditioned medium of MenSCs also increases the survival of mouse animals affected by sepsis.

Overall, the present invention offers an alternative method to treat infectious disease, especially involving sepsis. Since it is composed of mesenchymal stem cells present in menstrual fluid, the invention provides an ease access and repeated sampling in a non-invasive manner. Such attributes allow the rapid and cost-effective production of the treatment.

### BRIEF DESCRIPTION OF THE FIGURES

FIG. 1 shows the characterization of MenSCs and BMSCs.

Stem cells isolated from the menstrual fluid and bone marrow were characterized according to International Society for Cellular Therapy guidelines. A) MenSCs and BMSCs showed stem cell-like immunophenotypic markers. Fluorescence-activated cell sorting profile of representative cell samples. Blue and orange filled histograms denote the fluorescent profile of the indicated antigens in MenSCs and BMSCs, respectively; red histograms correspond to isotype-matched controls. B) MenSCs and BMSCs displayed mesodermal differentiation. Tri-lineage differentiation of representative MenSCs and BMSC samples. Cells were cultured with adipogenic, osteogenic, and chondrogenic induction media for 14-21 days and then stained with Oil Red O, Alizarin Red, and Safranin O staining, respectively. Scale bar: 200 μm. BMSCs bone marrow-derived mesenchymal stem cells, MenSCs menstrual derived mesenchymal stem cells.

FIG. 2 shows that MenSCs exert an important in vitro anti-microbial effect, which is associated in part with the increased expression of hepcidin.

MenSCs and BMSCs were evaluated in their capacity to inhibit bacterial proliferation. A) Direct inhibition of the bacterial proliferation. Cells were cultured in direct contact with a bacterial mixture. After 6 hours, the CM was collected and plated on blood-agar plates. Values are expressed as CFUs per milliliter. B) Indirect inhibition of bacterial proliferation. CM of cells cultured with or without bacterial stimulation was collected and their antimicrobial activity was tested by the incubation with a bacterial mixture. Values are expressed as CFUs per milliliter. C) Relative expression of hepcidin. The mRNA expression levels of hepcidin under both basal and stimulated conditions are determined by qRT-PCR. D) Inhibition of hepcidin by hypoxia. The mRNA expression levels of hepcidin under normoxic and hypoxic culture conditions in basal and stimulated MenSCs are determined by qRT-PCR. E) Direct inhibition of the bacterial proliferation under hypoxic conditions. MenSCs were cultured in direct contact with a bacterial mixture under normoxic and hypoxic conditions for 24 hours. After 24 hours, the CM was collected and plated on blood-agar plates. Values are expressed as CFUs per milliliter. Data are presented as the mean±standard error. BMSCs bone marrow-derived mesenchymal stem cells, CFU colony-forming unit, CM conditioned medium, MenSCs menstrual derived mesenchymal stem cells, ns non-significant, qRT-PCR quantitative reverse transcription-polymerase chain reaction.

FIG. 3 shows the experimental design for the in vivo studies.

Diagram of the experimental design of (A) survival studies and (B) therapeutical effects studies of MenSCs administration in mice with CLP-induced sepsis. Abbreviations: MenSCs, menstrual derived mesenchymal stem cells; AB, antibiotics; CM, conditioned medium; CFU, colony forming units.

FIG. 4 shows that treatment with MenSCs alone or in combination with antibiotics improves survival and protects against multiorgan dysfunction in CLP-induced sepsis.

A) Survival curves in mice with polymicrobial sepsis and different treatments. C57BL6/J mice subjected to CLP-induced sepsis were randomized into five groups: sham (n=12), saline (n=16), antibiotics (AB) (n=22), MenSCs (n=21), and MenSCs+AB (n=22). Three hours post-surgery, mice were injected with saline (vehicle), AB and/or MenSCs. Survival percentages of untreated and treated mice after CLP are presented as a Kaplan-Meier survival curve. B) Serum concentration of alanine aminotransferase (ALT), aspartate aminotransferase (AST), glucose, alkaline phosphatase (ALP), and albumin after CLP-induced sepsis. Serum was isolated 24 hours post-surgery and treatment administration (sham, n=3; saline, n=5; AB, n=4; MenSCs, n=4; MenSCs+AB, n=4), and the concentrations of biomarkers of liver function and total glucose were determined. Dot plots represent individual values, horizontal bars represent mean values, and vertical bars represent standard error values. C) Lung histology after CLP-induced sepsis. At 40 hours post-surgery and treatment infusion, lungs were collected, fixed, embedded in paraffin, and stained with hematoxylin-eosin. Pictures are representative images of lungs from the different experimental groups. AB antibiotics, CLP cecal ligation puncture, MenSCs menstrual derived mesenchymal stem cells.

FIG. 5 shows that MenSCs modulate the response of the host immune system to sepsis. At 24 and 40 hours after CLP-induced sepsis and treatment administration, blood samples were obtained to determine the serum concentration of the inflammatory cytokines (a) TNF-α, (b) MCP-1, (c) IL-6, and (d) IL-10 using a Cytometric Bead Array and analyzed by flow cytometry (sham, n=6; saline, n=3-11; AB, n=4-6; MenSCs, n=5-6; MenSCs+AB, n=6). Histograms represent the mean±standard error. AB antibiotics, CLP cecal ligation and puncture, IL interleukin, MCP-1 monocyte chemoattractant protein-1, MenSC menstrual derived mesenchymal stem cells, ns non-significant, TNF-α tumor necrosis factor-alpha.

FIG. 6 shows that MenSCs in combination with antibiotics prevent the decrease of CD45+CD3+ and CD45+CD19+ lymphocyte levels after CLP-induced sepsis.

Blood samples were obtained 24 and 40 hours after administration of various treatments post-CLP, and specific lymphocyte subsets were determined by fluorescence-activated cell sorting. A) Graphs represent the percentage of CD45+CD3+ and CD45+CD19+ lymphocytes and the ratio CD45+CD3+/CD45+CD19 (sham, n=3-6; saline, n=3-10; AB, n=3-6; MenSCs, n=3-6; MenSCs+AB, n=3-6). Histograms represent the mean±standard error. B) Representative dot plots of specific lymphocyte subsets described in (a) at 40 hours post-induction of sepsis and administration of different treatments. AB antibiotics, CLP cecal ligation and puncture, LB B lymphocytes, LT T lymphocytes, MenSCs menstrual derived mesenchymal stem cells, ns non-significant.

FIG. 7 shows that MenSCs alone or in combination with antibiotic treatment promote bacterial clearance in CLP-induced sepsis in mice.

Blood, peritoneal fluid, and spleen were obtained 24 hours after administration of the different treatments (sham, n=6; saline, n=11; AB, n=6; MenSCs, n=5; MenSCs+AB, n=6). Bacterial loads were determined after incubating at 37° C. overnight and are expressed as CFUs per milliliter. A) Bacterial load in blood. B) Bacterial load in peritoneal fluid. C) Bacterial load in spleen. Data are presented as mean±standard error. AB antibiotics, CFU colony-forming unit, CLP cecal ligation and puncture, MenSCs menstrual derived mesenchymal stem cells, ns non-significant.

FIG. 8 indicates that septic mice show higher retention of the injected MenSCs in the peritoneal cavity in comparison with healthy control mice.

MenSCs were labelled with PKH-26 and analyzed by flow cytometry. A) Fluorescence-activated cell sorting plot showing a 95% labelling efficiency. Around 2 Å^{˜}106 cells were injected intraperitoneally in septic or healthy mice. B) Histogram and representative flow cytometry plots showing the percentage of remaining cells in peritoneal lavage at 24 hours post-injection. CLP cecal ligation puncture, MenSCs menstrual derived mesenchymal stem cells.

FIG. 9 shows that MenSCs CM improves the survival in CLP-induced sepsis in mice. C57BL6/J mice with CLP-induced sepsis were randomized in five groups: sham (n=12), saline (n=16), antibiotics (AB) (n=22), MenSCs CM (n=16), and MenSC CM+AB (n=22). After 3 hours post-sepsis induction, mice were injected with saline (vehicle), AB and/or MenSC CM. Survival percentages of untreated and treated mice are represented as a Kaplan-Meier survival curve. AB antibiotics, CFU colony-forming unit, CLP cecal ligation and puncture, CM conditioned medium, MenSCs menstrual derived mesenchymal stem cells.

### DESCRIPTION

### Definitions

In the context of the present invention, the term “antibiotic” refers to an antimicrobial drug that has the capacity to kill (biocidal activity) or inhibit the growth (biostatic activity) of bacteria. Antibiotics that are sufficiently nontoxic to the host are used as chemotherapeutic agents in the treatment of infectious diseases.
In the context of the present invention, the term “Mesenchymal Stem Cells (MSCs)” refers to multipotent mesoderm-derived progenitor cells. They have the capacity to differentiate into cells that compose adipose, bone, cartilage, and muscle tissue. The minimal criteria set by The International Society for Cellular Therapy in assuring MSC identity by using CD70, CD90, and CD105 as positive markers and CD34 as a negative marker.
In the context of the present invention, the term “MenSCs” is understood as stem cells, mesenchymal stem cells, isolated from the menstrual fluid of woman that are in fertile ages. These cells show spindle-shape morphology, stem cell-like phenotypic markers: MenSCs express CD105, CD44, CD73, CD90 and HLA-ABC, but showed negative expression for CD45, CD34, CD14 and HLA-DR, and mesodermal lineage differentiation under specific protocols of laboratory.
In the context of the present invention, the term “cell culture” refers to the growth of cells in a medium in vitro. In such a culture, the cells proliferate, but they do not organize into tissue per se.
In the context of the present invention, the term “culture medium” is recognized in the art, and refers generally to any substance or preparation used for the cultivation of living cells.
In the context of the present invention, the term “conditioned medium (CM)” is recognized in the art, and refers to the culture medium and the components in suspension in the culture medium surrounding the cells while being cultured.
In the context of the present invention, the term “infectious disease” refers to disease caused by the invasion and multiplication of microorganisms such as bacteria, viruses, and parasites that are not normally present in the body. They are transmitted from person to person by direct or indirect contact. An infection may remain localized, or it may spread through the blood or lymphatic vessels to become systemic.
In the context of the present invention, the term “sepsis” refers to a specific reaction of the body to an infection insult, characterized by a hyper-immune response. It results in an intense surge of cytokines that leads to hypotension, multiple organ failure and sometimes to death. It is often accompanied by a state of relative immune paralysis caused by apoptosis of immune cells and high level of anti-inflammatory cytokines that inhibit lymphocytes and macrophages and suppress the production of pro-inflammatory cytokines. This immune paralysis is thought to cause the delayed mortality seen in some septic patients, due to their incapacity to eliminate the infection. The balance between hyper-immune response and immune paralysis varies with the patients and with the course of the illness within the same patient.
In the context of the present invention, the term “pharmaceutical composition” refers to a mixture containing one or several specific substances intended to furnish pharmacological activity or to otherwise have direct effect in the diagnosis, cure, mitigation, treatment or prevention of disease, or to have direct effect in restoring, correcting or modifying physiological functions in human beings. It preferably also contains a carrier.
In the context of the present invention, the term “carrier” refers to a pharmaceutically acceptable material, composition or vehicle, liquid or solid, diluent, excipient, or solvent encapsulating material, involved in carrying or transporting a specific compound from one organ, or portion of the body, to another organ, or portion of the body. It allows the delivery of the specific compound in a specific organ, or portion of the body. Each carrier must be acceptable in the sense of being compatible with the other ingredients of the formulation and not injurious to the patient.
Kit of parts, as used herein, shall encompass an entity of physically separated components, which are intended for individual use, but in functional relation to each other.
In the context of the present invention, the group “sham” refers to a group subjected to a faked surgical intervention that omits the step thought to be therapeutically necessary. Sham surgery serves an analogous purpose to placebo drugs, neutralizing biases such as the placebo effect.
In the context of the present invention, “T cell” or “T lymphocyte” refers to a type of lymphocyte (a subtype of white blood cell) that plays a central role in cell-mediated immunity. T cells can be distinguished from other lymphocytes, such as B cells and natural killer cells, by the presence of a T-cell receptor on the cell surface. T cells originate in the bone marrow and mature in the thymus. In the thymus, T cells multiply and differentiate into helper, regulatory, or cytotoxic T cells or become memory T cells. They are then sent to peripheral tissues or circulate in the blood or lymphatic system. Once stimulated by the appropriate antigen, helper T cells secrete chemical messengers called cytokines, which stimulate the differentiation of B cells into plasma cells (antibody-producing cells). Regulatory T cells act to control immune reactions, hence their name. Cytotoxic T cells, which are activated by various cytokines, bind to and kill infected cells and cancer cells.
In the context of the present invention, the term “B cells” or “B Lymphocyte” are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. B cells, unlike the other two classes of lymphocytes, T cells and natural killer cells, express B cell receptors (BCRs) on their cell membrane. BCRs allow the B cell to bind a specific antigen, against which it will initiate an antibody response.
In the context of the present invention, the term “CD19” refers to the Cluster of Differentiation 19. It is a protein found on the surface of B-cells.
In the context of the present invention, the term “CD3” refers to the Cluster of Differentiation 3. It is a T-cell co-receptor that helps to activate the cytotoxic T-Cell. The CD3 antigen is found bound to the membranes of all mature T-cells, and in virtually no other cell type, although it does appear to be present in small amounts in Purkinje cells. This high specificity, combined with the presence of CD3 at all stages of T-cell development, makes it a useful immunohistochemical marker for T-cells in tissue sections.
In the context of the present invention, the term “CD45” refers to the cluster of Differentiation 45. It is a protein present in all leukocytes, and more intensely expressed in lymphocytes B and T.
In the context of the present invention, the term “Alanine aminotransferase (ALT)” refers to a transaminase enzyme. It is also called alanine aminotransferase (ALAT) and was formerly called serum glutamate-pyruvate transaminase (SGPT) or serum glutamic-pyruvic transaminase (SGPT). ALT is found in plasma and in various body tissues, but is most common in the liver. It catalyzes the two parts of the alanine cycle. ALT catalyzes the transfer of an amino group from L-alanine to α-ketoglutarate, the products of this reversible transamination reaction being pyruvate and L-glutamate. ALT is commonly measured clinically as a part of a diagnostic evaluation of hepatocellular injury, to determine liver health.
In the context of the present invention, the term “aspartate transaminase (AST)” or aspartate aminotransferase, also known as AspAT/ASAT/AAT or serum glutamic oxaloacetic transaminase (SGOT), refers to a pyridoxal phosphate (PLP)-dependent transaminase enzyme. AST catalyzes the reversible transfer of an α-amino group between aspartate and glutamate and, as such, is an important enzyme in amino acid metabolism. AST is found in the liver, heart, skeletal muscle, kidneys, brain, and red blood cells. Serum AST level, serum ALT (alanine transaminase) level, and their ratio (AST/ALT ratio) are commonly measured clinically as biomarkers for liver health.
In the context of the present invention, “Glucose” refers to a sugar with the molecular formula C₆H₁₂O₆. Glucose circulates in the blood of animals as blood sugar. During sepsis patients, an uncontrollable inflammatory response can lead to many kinds of metabolic derangements. One such metabolic derangement is hyperglycemia, or high level of glucose in the blood.
In the context of the present invention, “albumin” refers to a protein made by the liver. A serum albumin test measures the amount of this protein in the clear liquid portion of the blood. Determining the albumin levels in blood can help determine if a patient has liver disease.
In the context of the present invention, “Alkaline phosphatase (ALP)” refers to a group of enzymes that catalyzes the hydrolysis of phosphate esters in an acidic environment. They have a low substrate specificity. Alkaline phosphatases are present in many human tissues, including bone, intestine, kidney, liver, placenta and white blood cells. Damage to these tissues causes the release of ALP into the bloodstream. Elevated levels can be detected through a blood test. Elevated alkaline phosphatase is most commonly caused by liver disease or bone disorders.
In the context of the present invention “TNF-α” refers to Tumor necrosis factor (TNF, tumor necrosis factor alpha, TNFα, cachexin, or cachectin). It is a cell signaling protein (cytokine) involved in systemic inflammation and is one of the cytokines that make up the acute phase reaction. It is produced chiefly by activated macrophages, although it can be produced by many other cell types such as T lymphocytes, NK cells, neutrophils, mast cells, eosinophils, and neurons. The primary role of TNF is in the regulation of immune cells. TNF, being an endogenous pyrogen, is able to induce fever, apoptotic cell death, cachexia, inflammation and to inhibit tumorigenesis and viral replication and respond to sepsis via IL1 & IL6 producing cells.
In the context of the present invention, “monocyte chemoattractant protein 1 (MCP1)”, or the chemokine (C-C motif) ligand 2 (CCL2), or small inducible cytokine A2, refers to a small cytokine that belongs to the CC chemokine family. MPC1 recruits monocytes, memory T cells, and dendritic cells to the sites of inflammation produced by either tissue injury or infection and in some cases, it perpetuates the inflammation.
In the context of the present invention, “Interleukin 6 (IL-6)” refers to an interleukin secreted by T cells and macrophages to stimulate immune response, e.g. during infection and after trauma, especially burns or other tissue damage leading to inflammation. It acts both as a pro-inflammatory cytokine and an anti-inflammatory myokine. Osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1ra and IL-10.
In the context of the present invention, “Interleukin 10 (IL-10)”, also known as human cytokine synthesis inhibitory factor (CSIF), refers to an anti-inflammatory cytokine. It is primarily produced by monocytes and, to a lesser extent, lymphocytes, namely type 2 T helper cells (T_{H}2), mast cells, CD4⁺CD25⁺Foxp3⁺ regulatory T cells, and in a certain subset of activated T cells and B cells.

### DETAILED DESCRIPTION OF THE INVENTION

MenSCs are stem cells, obtained from the menstrual fluid of women that are in fertile ages. MSCs derived from bone marrow have been shown to inhibit bacterial growth in co-culture (Anna Krasnodembskaya et al., Antibacterial Effect of Human Mesenchymal Stem Cells Is Mediated in Part from Secretion of the Antimicrobial Peptide LL-37. Stem cells 2010).

However, in the present invention, inventors show that conditioned medium (CM) obtained from non-stimulated MenSCs cell culture has a higher inhibitory effect on bacterial growth than that obtained from non-stimulated BMSCs (FIG. 2B). This indicates that MenSCs secrete a soluble factor to the medium that inhibits bacterial growth more efficiently than BMSCs. Furthermore, inventors identified Hepcidin as a very likely factor to mediate this effect (FIG. 2C-E).

In vivo experiments also showed the capacity of MenSCs to treat bacterial infections. For in vivo experiments, inventors used a CLP-induced sepsis model, wherein the cecum of a mouse is incised, exposed and ligated in the middle portion, punctured once, and returned to the peritoneal cavity. This surgery causes a polymicrobial sepsis. In the case of sham-operated mice, the cecum was exposed and ligated but not punctured (FIG. 3). Three hours post-CLP, animals where treated with a saline solution, an antibiotic (AB), MenSCs or MenSCs together with AB. At 24 hours post-CLP, as expected, animals treated with AB showed a significant reduction of bacterial concentration in blood, peritoneal fluid and spleen. Interestingly, the concentration of bacteria in blood was even lower in animals treated with MenSC or MenSC and AB, indicating that MenSC reduce the systemic bacterial proliferation, even more efficiently than AB (FIG. 7).

Additionally, inventors show that administration of MenSC to CLP-induced sepsis model increases the survival rates, and in combination with AB, even more than if administrating ABs alone. Importantly, when adminstrating MenSC in combination with AB, the survival rates are very similar to the sham group, indicating that, MenSCs, and preferably MenSC in combination with an antibiotic, are very suitable to increase survival of patients suffering from a severe infection, such as those involving sepsis.

Based on these results, a first aspect of the invention refers to a pharmaceutical composition comprising mesenchymal stem cells from menstrual fluid (MenSCs) for use in the treatment of an infection caused by microorganisms selected from the list consisting of bacteria, fungi, or parasites.

As shown in FIG. 5, treatment of CLP-induced mice with MenSCs showed a clear modulation of the inflammatory response at 40 h post-CLP. In this sense, CLP surgery induced an increase in the levels of different cytokines (TNF-α, IL6, MCP1, and IL10), and treatment with MenSCs, alone or in combination with ABs, brought cytokines to levels similar the sham group, similarly or even more efficiently than the treatment with ABs alone (FIGS. 5 A-D). Overall, results indicate that MenSCs, alone or in combination with ABs, can regulate the cytokines levels of patients with altered levels of pro- and anti-inflammatory cytokines as consequence of an infection. If not treated, this dysregulation of pro- and anti-inflammatory cytokines would lead to widespread inflammation and, likely to blood clotting.

Additionally, as shown in FIG. 6b, the CLP mice show a decrease in the levels of T and B lymphocytes, potentially leading to an immune paralysis. However, treatment with MenSCs restores the levels of T and B lymphocytes, similar to those of the sham group. Thus, a treatment with MenSCs has the potential to rescue a patient from an immune paralysis as consequence of an infection.

Finally, as a consequence of the above mentioned reactions of the organism to a severe infection, many patients show liver dysfunction. Interestingly, treatment of CLP mice with MenSCs restores the levels of several enzymes used as indicators of liver function. For instance, ALT, AST and ASP levels were increased in CLP mice, and upon treatment with MenSCs, alone or in combination with AB, the enzyme levels got closer to the ones of the Sham group (FIG. 4B). Reduced levels of albumin are also indicators of liver dysfunction, and upon treatment with MenSCS, albumin levels were restored. Low levels of glucose is also a commonly altered parameter in patients suffering the above mentioned type of infections, and again, treatment with MenSCS, alone or in combination with AB, helped to reach the glucose levels observed in the Sham group (FIG. 4B). Overall, experiments show that MenSCS can be used as a pharmaceutical composition for use in an infection characterized by a hyperimmune response of the host organism, followed by an immune paralysis and accompanied by the unbalance between the pro and anti-inflammatory cytokines leading to widespread inflammation and blood clotting.

Therefore, in a preferred embodiment of the first aspect of the invention, the pharmaceutical composition is for use in an infection characterized by a hyper immune response of the host organism, followed by an immune paralysis and accompanied by the unbalance between the pro and anti-inflammatory cytokines leading to widespread inflammation and blood clotting.

Most infections leading to a hyper immune response of the host organism, followed by an immune paralysis and accompanied by the unbalance between the pro and anti-inflammatory cytokines leading to widespread inflammation and blood clotting are caused by the following bacteria: Staphylococcus aureus (S. aureus), Streptococcus pyogenes (S. pyogenes), Streptococcus pneumoniae (S. pneumoniae), Klebsiella spp., Escherichia coli (E. coli), and Pseudomonas aeruginosa (P. aureginosa).

Therefore, another preferred embodiment of the first aspect of this invention is the pharmaceutical composition for use in the treatment of an infection caused by bacteria selected from the list consisting of Staphylococcus aureus (S. aureus), Streptococcus pyogenes (S. pyogenes), Streptococcus pneumoniae (S. pneumoniae), Klebsiella spp., Escherichia coli (E. coli), and Pseudomonas aeruginosa (P. aureginosa).

Examples also show that, in several regards, MenSc in combination with ABs show an increased therapeutic effect in comparison to MenSCs or ABs alone. For instance, the survival rates of CLP mice increases upon treatment with MenSCs, but only a combined treatment of MenSCS with AB allows to reaching survival rates similar to the sham group. Importantly, these survival rates are higher than when administrating only one of the treatments (MenSCs or ABs) separately (FIG. 4A). Similarly, in combination with AB, MenSCs seem to help to recover liver function slightly better than MenSc or AB alone. Indeed, CLP mice treated with MenScs in combination with AB show a slight (but not negligible) improvement of the levels of some biochemical markers of liver function, when compared with CLP mice treated only with AB or only with MenSCs (FIG. 4b). Therefore, another preferred embodiment of the first aspect of the present invention is that the pharmaceutical composition further comprises one or more antibiotics. The antibiotics commonly used to treat an infection leading to a body reaction such as the one described above can, for example, be selected from the following list of antibiotic families consisting of: carbapenems, penicillin, cephalosporins, glycopeptides, lipopeptide, monobactams, oxazolidinones, quinolones/fluoroquinolone, tetracyclines or any combinations thereof. Therefore, another preferred embodiment of the first aspect of the present invention is the pharmaceutical composition further comprises an antibiotic selected from the list consisting of carbapenems, penicillin, cephalosporins, glycopeptides, lipopeptide, monobactams, oxazolidinones, quinolones/fluoroquinolone, tetracyclines or any combination thereof.

More particularly, the antibiotics can be selected from the list consisting of: Enrofloxacin, Imipenem/cilastatin, Meropenem, Piperacillin and tazobactam, Ampicillin and sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin and Tigecycline. Therefore, another preferred embodiment of the first aspect of the present invention is the pharmaceutical composition further comprising one or more antibiotics, wherein the one or more antibiotics are selected from the list consisting of Enrofloxacin, Imipenem/cilastatin, Meropenem, Piperacillin and tazobactam, Ampicillin and sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin and Tigecycline.

A second aspect of the invention refers to a kit of parts comprising at least two components, recipients or vials A and B, wherein component, recipient or vial A comprises MenSCs and wherein component, recipient or vial B comprises antibiotics of any of the following families: carbapenems, penicillin, cephalosporins, glycopeptides, lipopeptide, monobactams, oxazolidinones, quinolones/fluoroquinolone, tetracyclines or any combination thereof.

Example 2 shows that the CM of MenSCs can be used to inhibit bacterial growth and importantly, that CM of non-stimulated MenSCs is more effective than that of BMSCs (FIG. 2B). Survival rates of CLP mice treated with MenSCs CM, alone or in combination with AB, also improved compared to non-treated animals (FIG. 9). Therefore, a third aspect of the present invention is a pharmaceutical composition comprising the CM of MenSCs for use in the treatment of an infection caused by microorganisms selected from the list consisting of bacteria, fungi or parasites.

CLP surgery in mice induce a poly-microbial sepsis reaction, which is characterized by a hyper-immune response of the host organism, followed by an immune paralysis and accompanied by the unbalance between the pro and anti-inflammatory cytokines leading to widespread inflammation and blood clotting. Since the CM of MenSCs improves the survival rates of CLP mice, a preferred embodiment of the third aspect of the present invention is the pharmaceutical composition wherein the composition is for use in an infection characterized by a hyperimmune response of the host organism, followed by an immune paralysis and accompanied by the unbalance between the pro and anti-inflammatory cytokines leading to widespread inflammation and blood clotting.

Most infections leading to the above mentioned reaction of the body are caused by the following bacteria: Staphylococcus aureus (S. aureus), Streptococcus pyogenes (S. pyogenes), Streptococcus pneumoniae, Klebsiella spp., Escherichia coli (E. coli), and Pseudomonas aeruginosa (P. aureginosa). Therefore, another preferred embodiment of the third aspect of the present invention is the pharmaceutical composition for use in the treatment of an infection caused by bacteria selected from the list consisting of Staphylococcus aureus (S. aureus), Streptococcus pyogenes (S. pyogenes), Streptococcus pneumoniae (S. pneumoniae), Klebsiella spp., Escherichia coli (E. coli), and Pseudomonas aeruginosa (P. aureginosa).

In combination with AB, the CM of MenSCs can improve the survival rates of the CLP mice (FIG. 9). Therefore, another preferred embodiment of the third aspect of the present invention is the pharmaceutical composition further comprising one or more antibiotics. The antibiotics commonly used to treat an infection leading to a body reaction such as the one described above are from the following list of antibiotic families: carbapenems, penicillin, cephalosporins, glycopeptides, lipopeptide, monobactams, oxazolidinones, quinolones/fluoroquinolone, tetracyclines or any combination thereof. Therefore, another preferred embodiment of the third aspect of the present invention is the pharmaceutical composition further comprising one or more antibiotics wherein the one or more antibiotics are selected from the list consisting of carbapenems, penicillin, cephalosporins, glycopeptides, lipopeptide, monobactams, oxazolidinones, quinolones/fluoroquinolone, tetracyclines or any combination thereof.

More particularly, the antibiotics can be selected from the list consisting of: Enrofloxacin, Imipenem/cilastatin, Meropenem, Piperacillin and tazobactam, Ampicillin and sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin and Tigecycline. Therefore, another preferred embodiment of the third aspect of the present invention is the pharmaceutical composition further comprising one or more antibiotics wherein the one or more antibiotics are selected from the list consisting of Enrofloxacin, Imipenem/cilastatin, Meropenem, Piperacillin and tazobactam, Ampicillin and sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin and Tigecycline.

Another preferred embodiment of the third aspect of the present invention is a kit of parts comprising at least two components, recipients or vials A and B, wherein component, recipient or vial A comprises the CM of MenSCs and wherein component, recipient or vial B comprises antibiotics of the following families: carbapenems, penicillin, cephalosporins, glycopeptides, lipopeptide, monobactams, oxazolidinones, quinolones/fluoroquinolone, tetracyclines or any combination thereof.

Further embodiments of the invention are disclosed as follows:
1—A pharmaceutical composition comprising mesenchymal stem cells from menstrual fluid (MenSCs) for use in the treatment of an infection caused by microorganisms selected from the list consisting of bacteria, fungi, or parasites.
2—The pharmaceutical composition for use according to claim 1, wherein the composition is for use in an infection characterized by a hyperimmune response of the host organism, followed by an immune paralysis and accompanied by the unbalance between the pro and anti-inflammatory cytokines leading to widespread inflammation and blood clotting.
3—The pharmaceutical composition according to claim 2 for use in the treatment of an infection caused by bacteria selected from the list consisting of Staphylococcus aureus (S. aureus), Streptococcus pyogenes (S. pyogenes), Streptococcus pneumoniae (S. pneumoniae), Klebsiella spp., Escherichia coli (E. coli), and Pseudomonas aeruginosa (P. aureginosa).
4—The pharmaceutical composition for use according to any of claims 2 to 3, further comprising one or more antibiotics.
5—The pharmaceutical composition for use according to claim 4, wherein the one or more antibiotics are selected from the list consisting of carbapenems, penicillin, cephalosporins, glycopeptides, lipopeptide, monobactams, oxazolidinones, quinolones/fluoroquinolone, tetracyclines or similar ones, drugs against mycobacteria.
6—The pharmaceutical composition for use according to claim 5, wherein the one or more antibiotics are selected from the list consisting of Enrofloxacin, Imipenem/cilastatin, Meropenem, Piperacillin and tazobactam, Ampicillin and sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin, Tigecycline.
7—A pharmaceutical composition comprising MenSCS and one or more antibiotics as defined in any of claims 4 to 6.
8—The pharmaceutical composition according to claim 7, wherein the list of active ingredients is selected from the list consisting of MenSCs and antibiotics of the following families: carbapenems, penicillin, cephalosporins, glycopeptides, lipopeptide, monobactams, oxazolidinones, quinolones/fluoroquinolone, tetracyclines or similar ones, drugs against mycobacteria.
9—A kit of parts comprising at least two components, recipients or vials A and B, wherein component, recipient or vial A comprises MenSCs and wherein component, recipient or vial B comprises antibiotics of the following families: carbapenems, penicillin, cephalosporins, glycopeptides, lipopeptide, monobactams, oxazolidinones, quinolones/fluoroquinolone, tetracyclines or similar ones, drugs against mycobacteria.
10—A pharmaceutical composition comprising the conditioned medium (CM) of MenSCs for use in the treatment of an infection caused by microorganisms selected from the list consisting of bacteria, fungi, or parasites.
11—The pharmaceutical composition for use according to claim 10, wherein the composition is for use in an infection characterized by a hyperimmune response of the host organism, followed by an immune paralysis and accompanied by the unbalance between the pro and anti-inflammatory cytokines leading to widespread inflammation and blood clotting.
12—The pharmaceutical composition according to claim 11 for use in the treatment of an infection caused by bacteria selected from the list consisting of Staphylococcus aureus (S. aureus), Streptococcus pyogenes (S. pyogenes), Streptococcus pneumoniae (S. pneumoniae), Klebsiella spp., Escherichia coli (E. coli), and Pseudomonas aeruginosa (P. aureginosa).
13—The pharmaceutical composition for use according to any of claims 11 to 12, further comprising one or more antibiotics.
14—The pharmaceutical composition for use according to claim 13, wherein the one or more antibiotics are selected from the list consisting of carbapenems, penicillin, cephalosporins, glycopeptides, lipopeptide, monobactams, oxazolidinones, quinolones/fluoroquinolone, tetracyclines or similar ones, drugs against mycobacteria.
15—The pharmaceutical composition for use according to claim 14, wherein the one or more antibiotics are selected from the list consisting of Enrofloxacin, Imipenem/cilastatin, Meropenem, Piperacillin and tazobactam, Ampicillin and sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin, Tigecycline.
16—A pharmaceutical composition comprising the CM from MenSCS and one or more antibiotics as defined in any of claims 13 to 15.
17—The pharmaceutical composition according to claim 16, wherein the list of active ingredients is selected from the list consisting of the CM of MenSCs and antibiotics of the following families: carbapenems, penicillin, cephalosporins, glycopeptides, lipopeptide, monobactams, oxazolidinones, quinolones/fluoroquinolone, tetracyclines or similar ones, drugs against mycobacteria.
18—A kit of parts comprising at least two components, recipients or vials A and B, wherein component, recipient or vial A comprises the CM of MenSCs and wherein component, recipient or vial B comprises antibiotics of the following families: carbapenems, penicillin, cephalosporins, glycopeptides, lipopeptide, monobactams, oxazolidinones, quinolones/fluoroquinolone, tetracyclines or similar ones, drugs against mycobacteria.

The following examples serve to illustrate the present invention but do not limit the same.

### EXAMPLES

Materials and Methods

After informed consent from the donors and ethical revision and approval from the ethics committees of Universidad de los Andes and Cells for Cells, menstrual fluids were collected from four healthy donors from 24 to 38 years old and bone marrow from three hip surgery patients from 60 to 72 years to old as described previously (Alcayaga-Miranda F et al., Characterization of menstrual stem cells: angiogenic effect, migration and hematopoietic stem cell support in comparison with bone marrow mesenchymal stem cells. Stem Cell Res Ther. 2015).

Briefly, to obtain and culture MenSCs samples were collected in a menstrual silicone cup (Mialuna®, Santiago, Chile). Menstrual blood samples were transferred into a 50 ml tube with 10 ml phosphate buffered saline (PBS) containing 0.25 mg/ml amphotericin B, penicillin 100 IU, streptomycin 100 mg/ml and 2 mM ethylenediaminetetraacetic acid (EDTA) (all from Gibco, Paisley, UK). Menstrual blood mononuclear cells were separated by Ficoll-Paque Plus (GE Healthcare, Amersham, UK) (1.077 g/ml) density gradient according to the manufacturer's instructions and washed in PBS. BM-MSCs were grown under the same conditions as MenSCs.

Cell Culture

MSCs were cultured as described previously (Alcayaga-Miranda F et al., Characterization of menstrual stem cells: angiogenic effect, migration and hematopoietic stem cell support in comparison with bone marrow mesenchymal stem cells. Stem Cell Res Ther. 2015). In brief, MSCs were cultured in Dulbecco's modified Eagle's medium (DMEM) (Invitrogen, part of Thermo Fisher Scientific, Waltham, Mass., USA) supplemented with 10% fetal bovine serum (FBS) (Gibco, Carlsbad, Calif., USA), 1% penicillin-streptomycin (Invitrogen), and 1% L-glutamine (Invitrogen). Cells were cultured at 37° C. with 5% CO2, routinely tested for mycoplasma (EZPCR Mycoplasma test kit; Biological Industries, Israel Beit-Haemek Ltd, Kibbutz Beit-Haemek, Israel) and cryopreserved in low passage (<3) until use. All cells were evaluated in their capacity to differentiate to adipocytes, osteocytes, and chondrocytes by using the StemPro Differentiation Kits (Gibco, Carlsbad, Calif., USA) in accordance with the instruction of the manufacturer. Immunophenotyping was performed by fluorescence-activated cell sorting (FACS) by using a FACSCanto II cytometer (BD Biosciences, San Jose, Calif., USA) after staining with monoclonal antibodies CD105, CD90, CD73, CD44, HLADR, CD34, and CD45 (all from BD Pharmingen, San Jose, Calif., USA) by using standard protocol. All experiments were performed by using cells in passage 4-8.

Antimicrobial Assay

Assessments of bacterial growth inhibition were performed as direct and indirect assays. Bacteria were isolated from mouse feces. For this, freshly harvested fecal material was diluted in 10 ml of sterile phosphatebuffered saline (PBS) and filtered through a 70-μm Cell Strainer (BD Falcon, Franklin Lakes, N.J., USA). Colonyforming unit (CFU) concentration was calculated by counting colonies plated on blood-agar plates after overnight incubation at 37° C. For direct inhibition assays, MSCs cultured in 24-well plates (2 Å^{˜}105 cells per well) in DMEM supplemented with 5% FBS and 1% L-glutamine were infected with 300 CFUs of a mix of bacteria and incubated for 6 or 24 hours at 37° C. with 5% CO2 (normoxia) or 24 hours at 37° C. with 1% O2 (hypoxia). Aliquots of the conditioned medium were taken from each well, serially diluted with sterile PBS, and plated on blood-agar plates. Colonies were counted after overnight incubation at 37° C. Antimicrobial activity of MSCs conditioned medium (CM) (non-stimulated CM condition) or from the co-culture above (stimulated CM condition) was tested as described previously with slight modifications (Krasnodembskaya A et al., Antibacterial effect of human mesenchymal stem cells is mediated in part from secretion of the antimicrobial peptide LL-37. Stem Cells. 2010). Briefly, MenSCs CM and BMSCs CM were collected, and the bacterial fraction was removed by passing the CM through a 0.22-μm filter (EdLab, New York, N.Y., USA). The filtered CM was centrifuged at 13,200 revolutions per minute for 10 minutes, and multiple freezing and thawing cycles were then performed (to eliminate any residual bacterial contamination). Prior to the experiments, samples were thawed on ice, and aliquots of 90 μl of the CM were transferred to a 96-well plate, inoculated with 100 CFUs of bacterial mix (in 10 μl of PBS), and incubated for 16 hours at 37° C. CFUs were then counted as described earlier.

mRNA Expression

Total RNAs was extracted by using the RNeasy kit (Qiagen, Marseille, France) in MSCs in basal condition or previously stimulated with 300 CFUs of bacterial mix. RNA (2 μg) was then reverse-transcribed by using the M-MLV enzyme (Fisher Scientific, Illkirch, France). The real-time quantitative polymerase chain reaction (PCR) was performed by using Stratagene Mx3000P (Agilent Technologies, Santa Clara, Calif., USA) with the following forward (F) and reverse (R) primers: hBD-1-F primer: 5′-GCCTCAGGTGGTAACTTTCTCA-3′, hBD-1-R primer: 5′-GATCGGGCAGGCAGAATAGAG-3′; hBD-2-F: 5′-GGAAGAAATGTCGCAGCCAAG-3′, hBD-2-R: 5′-CGATTCAGTAAGCTCTCATCCCAT-3′; hBD-3-F: 5′-TTATTGCAGAGTCAGAGGCGG-3′, hBD-3-R: 5′-TTTCTTCGGCAGCATTTTCGG-3′; hepcidin-F: 5′-CCCATGTTCCAGAGGCGAAG-3′, hepcidin-R: 5′-CTTGCAGCACATCCCACACT-3′. For LL-37 peptide mRNA quantification was determined by TaqMan gene expression assay (ID Hs00189038_m1; Applied Biosystems, Waltham, Mass., USA). All values were normalized to either GAPDH (GAPDH-F: 5′-GGTCTCCTCTGACTTGAACA-3′, GAPDH-R: 5′-GTGAGGGTCTCTCTCTTCCT-3′) or β 2M housekeeping gene (ID Hs00984230_m1; Applied Biosystems) and expressed as relative expression or fold change using the 2-ΔCT formula (Livak K J et al., Analysis of relative gene expression data using real-time quantitative PCR and the 2(−Delta Delta C(T)) Method. Methods. 2001).

Preparation of Conditioned Medium-Derived MenSCs

MenSCs were cultured in normal medium until 80% confluence was reached. After three washes with PBS, cells were cultured in serum-free DMEM supplemented with 1% penicillin-streptomycin and 1% L-glutamine at 37° C. with 5% CO2. After 72 hours post-culture, the supernatant was collected. The cellular debris were removed by centrifugation at 500 Å^{˜}g for 5 minutes at room temperature (RT). The supernatant was concentrated approximately 25- to 30-fold by ultrafiltration using Amicon Ultra centrifugal filters (Merck Millipore, Tullagreen, Ireland) with a membrane NMWL of 3 kDa in accordance with the instructions of the manufacturer. The concentrated medium was sterilized by filtration through a 0.22-μm syringe filter (Membrane Solutions LLC, Dallas, Tex., USA), and the protein concentration was determined by Bradford protein assay (Bio-Rad Laboratories, Hercules, Calif., USA). The mean protein concentration was 0.3±0.2 μg/ul. Equal volumes of serum free DMEM but without cells were used under the same conditions and served as negative control.

Polymicrobial CLP-Induced Sepsis

In vivo studies were performed at the Universidad de los Andes-Cells for Cells Animal Facility (Santiago, Chile) in accordance with the institutional guidelines for the care and use of laboratory animals in research, revised and approved by the Institutional Ethical Committee for animal experimentation. C57BL6/j mice (8 to 12 weeks old; Jackson Laboratories, Bar Harbor, Me., USA) received ad libitum access to food and water during the whole experimental procedure. CLP-induced sepsis was performed as described previously (Rittirsch D et al., Immunodesign of experimental sepsis by cecal ligation and puncture. Nat Protoc. 2009) with some modifications. Briefly, mice were anesthetized with inhaled Sevofluorane (Baxter, Guayama, Puerto Rico) by using an anesthetic vaporizer. After a 1-cm incision was performed, the cecum was exposed and ligated in the middle portion with 5-0 dafilon suture (Braun, Rubi, Spain), punctured once with an 18-gauge needle (in order to induce high-grade sepsis), and returned to the peritoneal cavity. The abdominal incision was sutured with catgut 5-0 (Braun, Bogota, Colombia), and the skin was closed with Histoacryl (Braun). In sham-operated mice, the cecum was exposed and ligated but not punctured. Immediately after the CLP procedure, all animals received pre-warmed fluid resuscitation with 50 ml/kg saline fluid by subcutaneous injection.

CM-Derived MSCs

Three hours after CLP-induced sepsis, mice were randomized and distributed into different experimental groups to receive single injections of saline (vehicle) (n=16), antibiotics (AB) (n=22), MenSCs (n=21), MenSCs+AB (n=22), MenSC CM (n=16), or MenSC CM+AB (n=22). MenSCs (7.5×10⁵ cells/mice) and MenSC CM (70 μg/300 μl) were inoculated via intra-peritoneal or intravenous injection, respectively, alone or with Enrofloxacin 5 mg/kg (Centrovet, Santiago, Chile). For Kaplan-Meier survival curves, animals were assessed after CLP-induced sepsis every 12 hours for 4 days. Fluid resuscitation and antibiotic therapy were administered by subcutaneous injections

immediately after the CLP procedure and repeated every 24 hours for 4 days. For the evaluation of the therapeutic effect, animals were sacrificed at 24-40 hours after CLP-induced sepsis, and total blood was collected by cardiac puncture for measurement of hematological and biochemical parameters and cytokine levels. The peritoneal cavity was washed, and lavage fluid was collected to determine the bacterial CFUs as previously described (Ray A, Dittel B N. Isolation of mouse peritoneal cavity cells. J Vis Exp. 2010). Liver, kidney, and lungs were removed, fixed in 10% formalin solution, and embedded in paraffin by standard methods. Samples were cut into 5-μm sections and stained with hematoxylin and eosin in accordance with standard protocols.

Biodistribution of MenSCs Injected in Septic Mice.

At 24 hours post-CLP procedures, 10⁷ MenSCs were labeled with PKH26 (Sigma-Aldrich, St. Louis, Mo., USA) in accordance with the protocol of the manufacturer. Labeling efficiency was 95% as validated by flow cytometry. Labelled MenSCs (2×10⁶ cells/mouse) were resuspended in 250 μl of PBS and injected intraperitoneally. Animals were euthanized at 24 hours post-injection, and different organs and fluids, including the spleen, heart, kidneys, lungs, liver, and intraperitoneal fluid, were recovered and incubated at 37° C. for 30 minutes with 250 U/ml of collagenase II and IV (Gibco). The single-cell suspensions obtained were resuspended into 400 μl of cytometry buffer (PBS 1×, bovine serum albumin (BSA) 0.2%, sodium azide %) and then analyzed by flow cytometry.

Bacterial Numbers in Blood, Peritoneal Cavity, and Spleen in CLP-Induced Sepsis

Total blood, peritoneal fluid, and homogenized spleen in sterile saline were plated in a 10-fold dilution on blood agar plates and incubated at 37° C. overnight, and the number of CFUs was determined.

Cytokine Multiplex Array Evaluation in CLP Mice

The levels of inflammatory cytokines-monocyte chemoattractant protein-1 (MCP-1), TNF-α, IL-6, and IL-8—in CLP mice serum samples were determined by using a commercial BD™ Cytometric Bead Array Mouse Inflammation Kit (BD Biosciences Pharmingen, San Diego, Calif., USA) in accordance with the instructions of the manufacturer. Analysis was carried out by flow cytometry, and the concentration (in picograms per milliliter) was determined by using FCAP Array software (BD Biosciences). The parameters were determined 24 and 40 hours after CLP surgery.

Blood Biochemical Analysis in CLP Mice

Plasma levels of blood urea nitrogen (BUN), bilirubin, alanine aminotransferase (ALT), aspartate aminotransferase (AST), alkaline phosphatase (ALP), albumin, amylase, and glucose were evaluated by using the Piccolo Xpress™ Chemistry Analyzer (Abaxis, Union City, Calif., USA) in accordance with the instructions of the manufacturer. Serum creatinine was determined by using a commercial kit (Valtek Diagnostics, Santiago, Chile) in accordance with the instructions of the manufacturer.

Lymphocyte Subset Analysis by FACS

Blood was collected by intracardiac puncture with EDTA as anticoagulant. The red blood cells were lysed with ACK Lysing Buffer (Gibco) in accordance with the instructions of the manufacturer. The collected cells were resuspended in 100 μl of FACS buffer (PBS 1×, 0.2% BSA, 0.01% sodium azide) and incubated for 20 minutes at 4° C. with the appropriate fluorescently labeled monoclonal antibody directed against lymphocyte surface markers (BD Biosciences), washed and resuspended in FACS buffer, and analyzed by the FACS Canto II cytometer by using the FACS Diva software (BD Biosciences). The viability was determined by using a LIVE/DEAD® Fixable dead cell stain kit (Invitrogen) in accordance with the protocol of the manufacturer. Approximately 20,000 gated events were assessed to determine the percentage of each subset: total lymphocytes (CD3+/APC), CD4+T lymphocytes (CD3+APC, CD4+/PE), CD8+T lymphocytes (CD3+/APC, CD8+/FITC), and total B lymphocytes (CD19+/FITC). The CD4/CD8 and CD3/CD19 ratios were calculated as the percentage of CD4+ Tcells divided by the percentage CD8+ T cells and the percentage of CD3+ T cells divided by the percentage CD19+ T cells.

Statistical Analysis

Data are expressed as mean±standard error. Comparisons of mortality were made by analyzing Kaplan-Meier survival curves and then log-rank test to assess for differences in survival. Mann-Whitney U test was used to evaluate the differences between groups. One-way analysis of variance followed by Tukey's post-test was used for analysis of multiple comparison groups. The numbers of samples per group (n) are specified in the figure legends. Statistical significance was set at *P<0.05, **P<0.01, and ***P<0.001.

### Results

### Example 1. Characterization of Mesenchymal Stem Cells Used in the Study

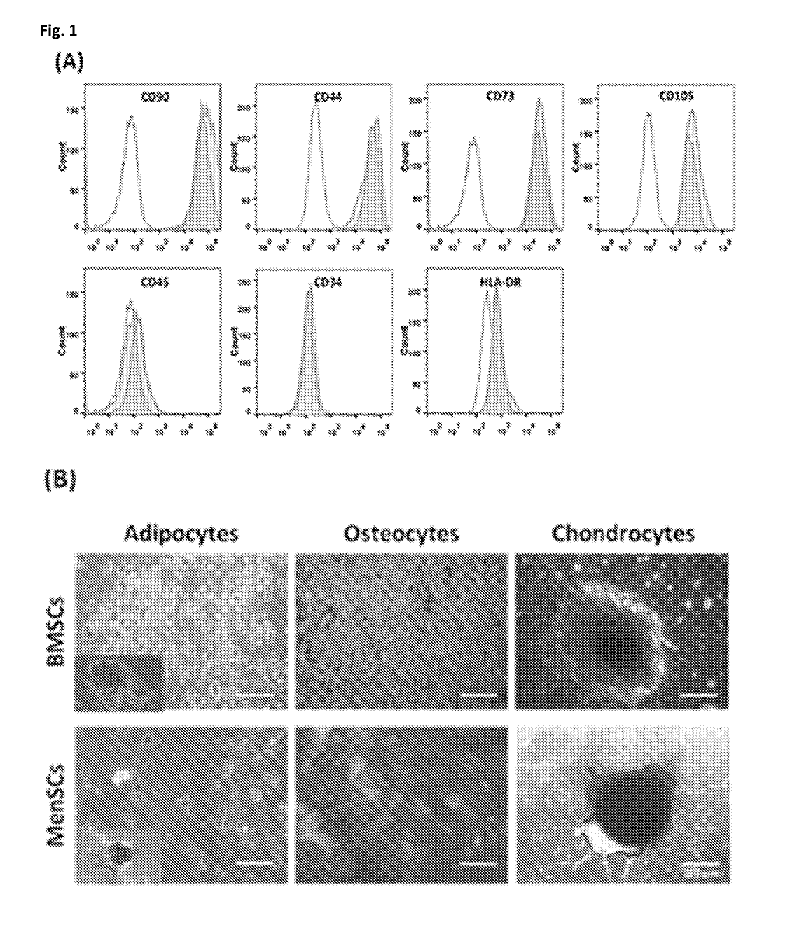

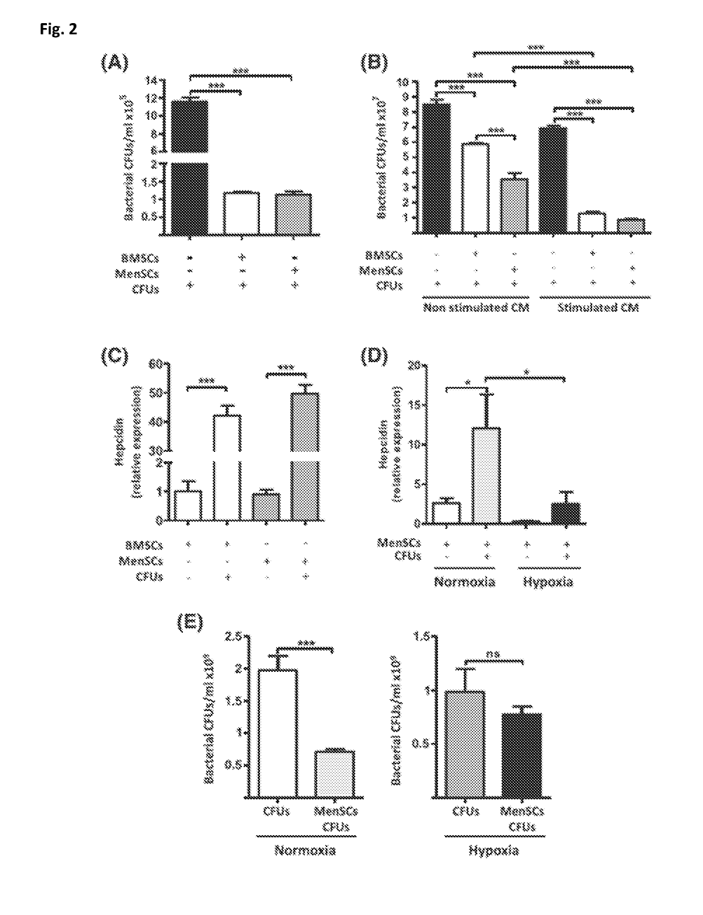

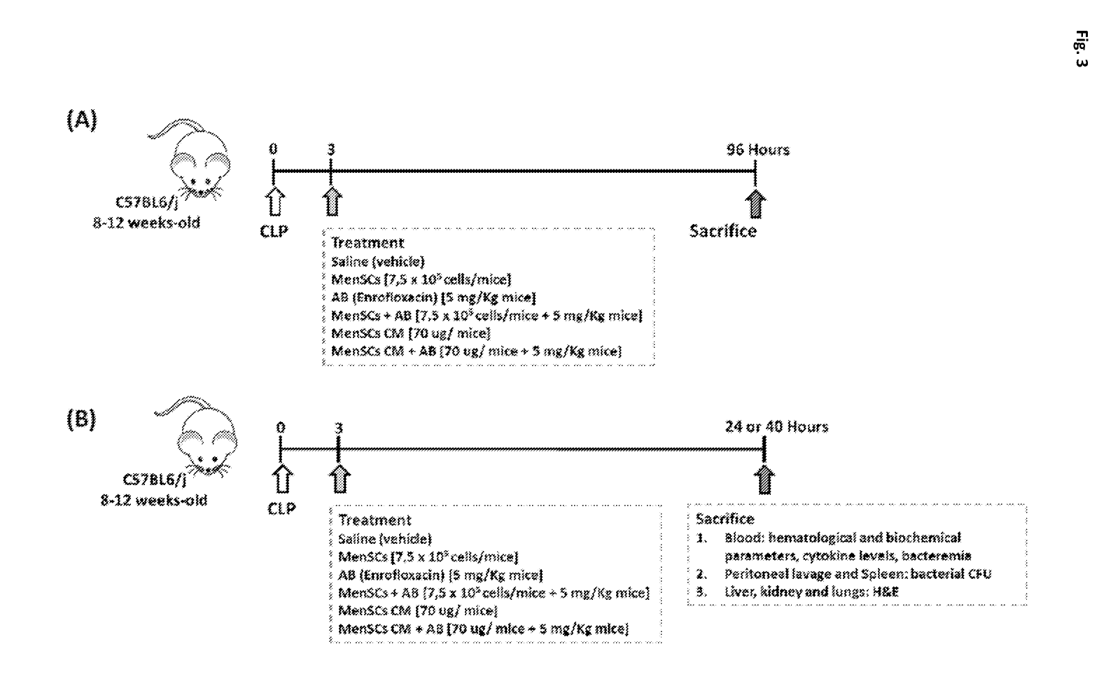

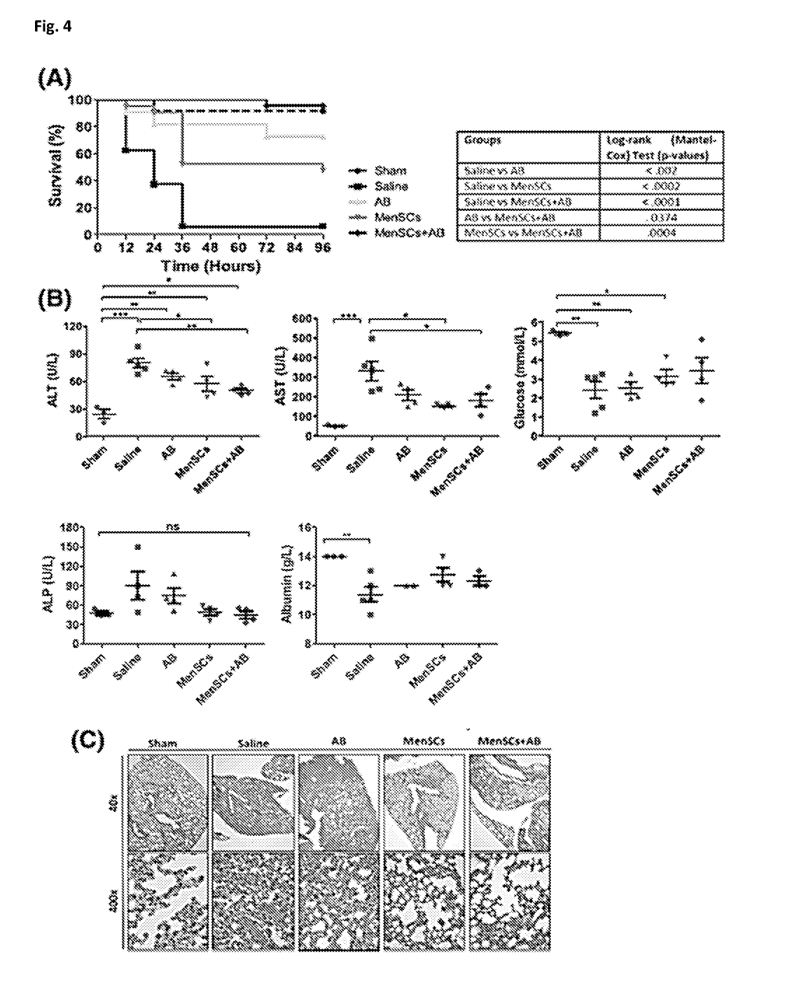

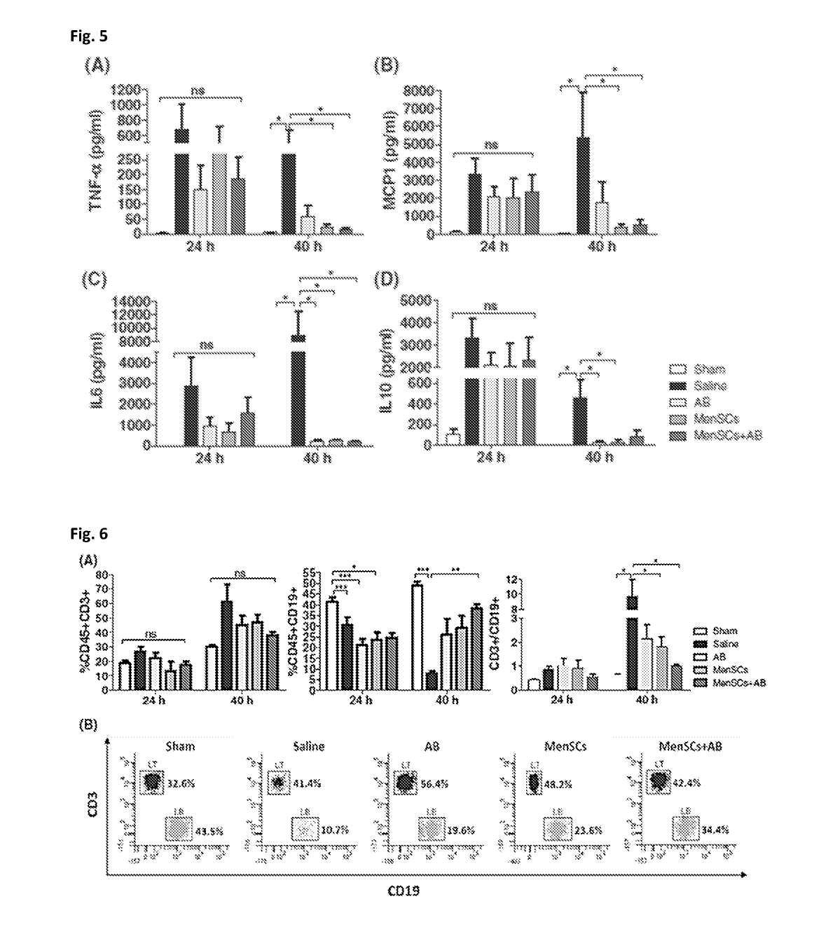

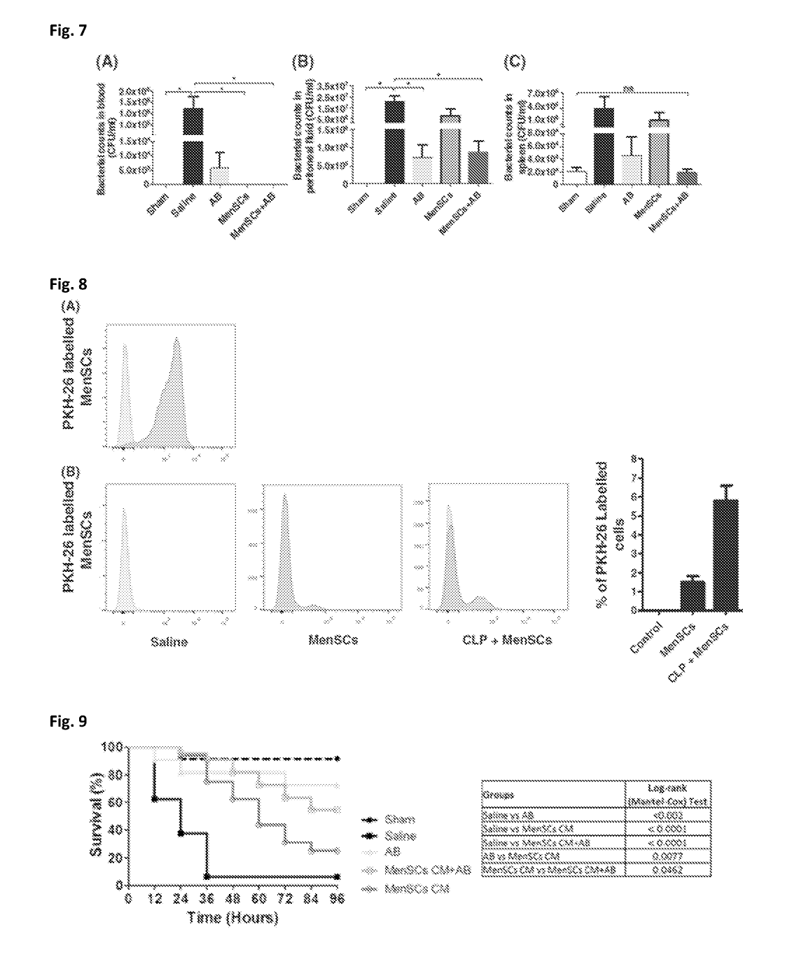

All MSCs used in this study showed adherence to plastic, expression of classic MSCs markers, and differentiation to osteoblasts, adipocytes, and chondroblasts (FIG. 1) in accordance with previously described criteria (Alcayaga-Miranda F et al., Characterization of menstrual stem cells: angiogenic effect, migration and hematopoietic stem cell support in comparison with bone marrow mesenchymal stem cells. Stem Cell Res Ther. 2015).

### Example 2. MenSCs Exert an In Vitro Anti-Bacterial Effect, Associated in Part with the Increased Expression of Hepcidin

The authors first compared the anti-microbial effect of MenSCs with respect to BMSCs to establish the comparative anti-microbial potency of MenSCs. For the direct assay, MSCs were incubated with a bacterial mixture for 6 hours. As shown in FIG. 2a, both MenSCs and BMSCs exerted a significant inhibition of the bacterial growth in comparison with control (P≤0.001). To determine whether this anti-bacterial effect is associated with the secretion of soluble factors, CM was studied (indirect assay) in accordance with published protocols (Krasnodembskaya A et al., Antibacterial effect of human mesenchymal stem cells is mediated in part from secretion of the antimicrobial peptide LL-37. Stem Cells. 2010). Whereas both non-stimulated and stimulated CM exhibited important anti-bacterial activity, the effect was increased when using stimulated CM (P≤0.001). Results also showed that MenSCs non-stimulated CM exert a greater inhibition of bacterial growth compared with BMSCs non-stimulated CM (P≤0.001) (FIG. 2b).

Taken together, these data suggest that inhibition of bacterial growth is associated with the secretion of soluble factors by MSCs and that, at least in in vitro conditions, MenSCs appear to be a better cell candidate for control of bacterial growth compared with BMSCs.

The inventors then analyzed the expression of different anti-microbial peptides (AMPS) under basal conditions and after bacterial stimulation. A low expression level of hepcidin was detected in both cell sources. The bacterial stimulation induced the increase of hepcidin expression up to 42- and 50-fold in BMSCs and MenSCs, respectively (P≤0.001) (FIG. 2c). Hypoxic culture conditions are known to inhibit the expression of hepcidin in hepatoma cells (Chalton T B et al., Hypoxia inhibits hepcidin expression in HuH7 hepatoma cells via decreased SMAD4 signaling. Am J Physiol Cell Physiol. 2011). Therefore, MenSCs were cultured for 24 hours in both normal and hypoxic conditions and in the presence or absence of the bacterial stimulus. Under hypoxia, the expression of hepcidin decreased in both conditions; however, statistical significance was obtained only for the stimulated MenSCs (P≤0.05), abrogating the effect of bacterial stimulus on hepcidin expression (FIG. 2d). Furthermore, the inhibition of hepcidin by the hypoxic conditions entailed a loss of the anti-microbial activity of MenSCs in comparison with normoxic conditions (FIG. 2e).

Overall, MenSCs have an anti-bacterial effect that is at least partially mediated by secretion of hepcidin to the media.

### Example 3. MenSCs Improve Survival of CLP-Induced Sepsis Model

Next, the inventors tested the therapeutic effect of MenSCs to treat sepsis, a specific reaction of the body upon an infection isult. They used a Murine model of polymicrobial septic peritonitis induced by cecal ligation and puncture (CLP) (FIG. 3). At 96 hours after different treatment, a significant increase in the survival rate of MenSCs+AB treatment group was observed in comparison with all the other experimental groups (saline, P≤0.0001; AB, P=0.0374; MenSCs, P=0.0004) as shown in FIG. 4a. The survival rate reached by MenSCs+AB group was 95% (21/22), while survival in the saline, AB, and MenSCs groups was 6% (1/16), 73% (16/22) and 48% (11/21), respectively. Both conventional AB and MenSCs treatments increased the survival of animals compared with saline (P≤0.002 and P≤0.0002, respectively). No differences were observed between AB group compared with the MenSCs group.

Results indicate that MenSC can improve the recovery of animal in sepsis, similarly to the treatment with antibiotics, and that in combination with AB, MenSC can save almost all the animals suffering from sepsis (at least at 96 h after the CLP induction).

### Example 4. MenSCs Improve Multiorgan Disfunction in CLP-Induced Sepsis Model

Sepsis lethality is associated with multiple organ failure. Therefore, to test if MenSCs have a therapeutic effect in animals in sepsis the inventors checked several biochemical indicators of hepatic, renal, and pancreatic function in serum samples. Liver function was markedly improved in animals that received MenSCs without or with AB (FIG. 4b). The concentrations of the liver enzymes alanine aminotransferase (ALT) and aspartate aminotransferase (AST) were detected at lower values in the MenSCs (ALT 57.25±8.107 U/l P≤0.05; and AST 153.5±5.299 U/l P≤0.05) and MenSCs+AB group (ALT 49.75±2.428 U/l P≤0.01; and AST 182.3±1.99 U/l P≤0.05) compared with the saline group (ALT 79.8±5.083 U/l and AST 331.2±49.29 U/l), suggesting that the protective or regenerative liver effect seems to be contributed mainly by the MenSCs. Although no difference was found in the concentration of alkaline phosphatase (ALP) enzyme, the analysis showed similar concentrations between sham (47.67±3.18 U/l), MenSCs (48.75±5.17 U/l), and MenSCs+AB (44.75±5.45 U/l) groups, in contrast to the increased levels observed in saline (90±21.47 U/l) and AB (74.25±12.03 U/l) groups (FIG. 4b). Altered blood glucose concentration is commonly observed in sepsis and is associated with adverse outcome (Peralta G et al., Altered blood glucose concentration is associated with risk of death among patients with community-acquired Gram-negative rod bacteremia. BMC Infect Dis. 2010). Decreased serum albumin levels are associated with acute-phase response of the liver (Ceciliani F et al., The systemic reaction during inflammation: the acute-phase proteins. Protein Pept Lett. 2002; Fulop A K. Genetics and genomics of hepatic acute phase reactants: a mini-review. Inflamm Allergy Drug Targets. 2007) and an increased capillary leakage (Fleck A et al., Increased vascular permeability: A major cause of hypoalbuminaemia in disease and injury. Lancet. 1985; Nicholson J P et la., The role of albumin in critical illness. Br J Anaesth. 2000). After MenSCs+AB treatment, the inventors observed a normalization in the blood glucose and albumin levels, indicating a recovery in the organ-wide metabolic failure. Specifically, animals from the MenSCs+AB group (3.475±0.6787 U/l) exhibited levels similar to animals from the Sham group (5.433±0.088 U/l), but animals from the saline (2.44±0.45 U/l), AB (2.55±0.31 U/l) and MenSCs (3.175±0.35 U/l) groups had a decrease in serum glucose concentration. The levels of serum albumin demonstrated a reduction in the saline group (2.440±0.4490 g/dl) compared with the sham group (5.433±0.088 g/dl P≤0.01), but an upward tendency was observed in groups of animals that received cells (FIG. 4b).

In addition, consistently with other reports (Mei S H et al., Mesenchymal stem cells reduce inflammation while enhancing bacterial clearance and improving survival in sepsis. Am J Respir Crit Care Med. 2010; Lee J W et al., Allogeneic human mesenchymal stem cells for treatment of E. coli endotoxin-induced acute lung injury in the ex vivo perfused human lung. Proc Natl Acad Sci USA. 2009) lung histology assessment revealed that MSCs treatment effectively prevented acute lung injury (FIG. 4c). In lungs of animals from the saline group, vast areas of atelectasis were observed with marked venular and capillary congestion with reduced airspaces and a moderate amount of septal leucocytes. In contrast, in all treated groups, the lungs showed irregular distended alveoli and isolated areas of atelectasis with moderate capillary congestion, irregular alveolar distension, and normal amounts of septal leukocytes.

Taken together, results indicate that MenSCs alone, or in combination with antibiotics, protect against multiorgan dysfunction in CLP-induced sepsis.

### Example 5. MenSCs Downregulates the Inflammatory Response In Vivo

To determine the effect of MenSCs on the immune response to infection, serum TNF-α, IL-6, MCP-1, and IL-10 were determined. Whereas at 24 hours post-CLP no differences in the serum levels of cytokines were detected, at 40 hours a clear modulation of the inflammatory response was observed in treated animals (FIG. 5). Specifically, a reduction in the TNF-α and MCP-1 serum concentration was observed in the MenSCs with and without AB groups in comparison with the saline group (P≤0.05) (FIG. 5a, b). Also, IL-6 serum levels were decreased in the AB, MenSCs, and MenSCs+AB groups in comparison with the saline group (P≤0.05) (FIG. 5c). Antibiotic and MenSCs groups showed a reduction in the levels of IL-10 in comparison with untreated mice (P≤0.05) (FIG. 5d).

Taken together, results show that animals receiving treatment showed modulation of the innate immune response, but more specifically, groups treated with MenSCs without or with AB presented a significant reduction in pro- and anti-inflammatory cytokines, suggesting that MenSCs can effectively rescue mice from the uncontrolled systemic inflammatory response after CLP-induced sepsis.

### Example 6. MenSCs in Combination with Antibiotics Prevent the Decrease of Lymphocyte Levels after CLP-Induced Sepsis

With the progression of sepsis, the adaptive immune system reduces its efficiency by an increase in anti-inflammatory mediators and loss of T and B cells via apoptosis [30, 31]. At 24 hours, increases of the distribution of CD45+CD3+CD8+ and CD4+/CD8+ were observed in the MenSCs group in comparison with the saline group (P≤0.05); In CD45+CD3+ cell populations, no difference between groups was detected at 24 and 40 hours post-CLP. However, a reduction in circulating CD45+CD19+ lymphocytes was observed at 24 hours in treated (P≤0.05; P≤0.001) and non-treated (P≤0.001) groups, showing an important recovery exclusively in treated animals at 40 hours, reaching a significant increase in the MenSCs+AB group (P≤0.01) (FIG. 6a) in comparison with untreated animals. In consequence, a reduction was also observed in the CD45+CD3+/CD45+CD19+ ratio in cell-treated groups in comparison with the untreated group (P≤0.05). Representative dot plots of the specific lymphocyte subsets at 40 hours after sepsis induction and administration of different treatments are shown in FIG. 6b. Altogether, no significant loss of T and B lymphocytes was observed in all treated groups, suggesting that MenSCs can effectively modulate the immune response in animals with sepsis without a severe immunosuppression.

### Example 7. MenScs Suppress the Systemic Bacterial Proliferation

Bacterial clearance was assessed in the blood, peritoneal fluids, and spleen of treated animals. At 24 hours post-CLP, animals that received MenSC treatment alone or in combination with antibiotics showed a lower bacterial load in blood compared with the untreated group (P≤0.05) (FIG. 7a), reaching values similar to those of the sham group (MenSCs 0.2±0.2 CFU/ml; MenSCs+AB 0.17±0.17 CFU/ml; and sham 0.17±0.17 CFU/ml). In peritoneal lavage, administration of AB alone or in combination with MenSCs decreased CFU counts in comparison with the saline group (P≤0.05) (FIG. 7b). In contrast, no differences in spleen were observed among different treatment groups (FIG. 7c).

Altogether, the results indicate that antibiotics contribute to the control of the bacterial growth in local sepsis (abdominal cavity) but that MenSCs appear to suppress the systemic bacterial proliferation.

### Example 8. Septic Mice Display Higher Retention of MenSCs in the Peritoneal Cavity

To assess the fate in the peritoneal cavity of the CLP mice, MenSCs were labeled with PKH-26 (FIG. 8a) and injected intraperitoneally in healthy and sepsis-induced mice. After 24 hours post-injection, the spleen, heart, kidneys, lungs, liver, and peritoneal fluid were collected to detect labeled cells by flow cytometry analysis. Although no labeled MenSCs were detected in the different analyzed organs, the injection site contained detectable levels of PKH-26+ cells. Furthermore, septic mice showed a fourfold increase in the retention of injected MenSCs in the peritoneal cavity in comparison with healthy control mice (FIG. 8b)

### Example 9. Conditioned Medium from MenSCs Improve Survival and Multiorgan Dysfunction in the CLP-Induced Sepsis Model

In line with the marked inhibition of bacterial growth observed in vitro (FIG. 2), the inventors tested whether the benefits of the MenSCs for the treatment of sepsis in vivo are also mediated by factors secreted by the MenSCs to the media. To this end, inventors evaluated the therapeutic effect of the MenSC CM in the CLP-induced sepsis model (FIG. 9). At 96 hours post-sepsis induction, MenSCs CM without or with AB increased survival compared with saline group (P<0.0001). The MenSC CM+AB group exhibited a larger increase in survival than that where MenSC CM was administered alone with survival rates of 55% (12/22) and 25% (4/16), respectively (P=0.0462). Interestingly, although the improvement in survival of the MenSCs CM+AB treatment group matched that observed in the MenSCs treatment group, it did not surpass that of the MenSCs+AB treatment.

Overall, results show that factors secreted to the CM have a therapeutical effect on animals suffering from sepsis.

1- A method for treating an infection in a subject in need thereof, the method comprising administering to the subject a pharmaceutical composition comprising mesenchymal stem cells from menstrual fluid (MenSCs), wherein the infection is caused by a microorganism selected from the group consisting of a bacterium, a fungus and a parasite, such that the infection in the subject is treated.

2- The method of claim 1, wherein the infection is characterized by a hyperimmune response of the host organism, followed by immune paralysis and accompanied by an imbalance of pro- and anti-inflammatory cytokines leading to widespread inflammation and blood clotting.

3- The method of claim 1, wherein the infection is caused by a bacterium belonging to a bacterial species selected from the group consisting of Staphylococcus aureus (S. aureus), Streptococcus pyogenes (S. pyogenes), Streptococcus pneumoniae (S. pneumoniae), Klebsiella spp., Escherichia coli (E. coli), and Pseudomonas aeruginosa (P. aureginosa).

4- The method of claim 1, wherein the pharmaceutical composition further comprises one or more antibiotic.

5- The method of claim 4, wherein the one or more antibiotic is selected from the group consisting of a carbapenem, penicillin, a cephalosporin, a glycopeptide, a lipopeptide, a monobactam, an oxazolidinone, a quinolone/fluoroquinolone, a tetracycline or an analog thereof and an antibiotic active against micobacteria.

6- The method of claim 5, wherein the one or more antibiotic is selected from the group consisting of Enrofloxacin, Imipenem/cilastatin, Meropenem, Piperacillin/tazobactam, Ampicillin/sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin and Tigecycline.

7- A pharmaceutical composition comprising MenSCS and one or more antibiotic selected from the group consisting of a carbapenem, penicillin, a cephalosporin, a glycopeptide, a lipopeptide, a monobactam, an oxazolidinone, a quinolones/fluoroquinolone, a tetracycline or an analog thereof and an antibiotic active against micobacteria.

8- The pharmaceutical composition of claim 7, wherein the one or more antibiotic is selected from the group consisting of Enrofloxacin, Imipenem/cilastatin, Meropenem, Piperacillin/tazobactam, Ampicillin/sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin and Tigecycline.

9- A kit comprising at least two components, recipients or vials A and B, wherein the component, recipient or vial A comprises MenSCs and wherein the component, recipient or vial B comprises an antibiotic selected from the group consisting of a carbapenem, penicillin, a cephalosporin, a glycopeptide, a lipopeptide, a monobactam, an oxazolidinone, a quinolone/fluoroquinolone, a tetracycline, an analog thereof and an antibiotic active against micobacteria.

10- A method for treating an infection in a subject in need thereof, the method comprising administering to the subject a pharmaceutical composition comprising conditioned medium (CM) of MenSCs, wherein the infection is caused by a microorganism selected from the group consisting of a bacterium, a fungus and a parasite, such that the infection in the subject is treated.

11- The method of claim 10, wherein the infection is characterized by a hyperimmune response of the host organism, followed by immune paralysis and accompanied by an imbalance of pro- and anti-inflammatory cytokines leading to widespread inflammation and blood clotting.

12- The method of claim 11, wherein the infection is caused by a bacterium belonging to a bacterial species selected from the group consisting of Staphylococcus aureus (S. aureus), Streptococcus pyogenes (S. pyogenes), Streptococcus pneumoniae (S. pneumoniae), Klebsiella spp., Escherichia coli (E. coli), and Pseudomonas aeruginosa (P. aureginosa).

13- The method of claim 11, wherein the pharmaceutical composition further comprises one or more antibiotics.

14- The method of claim 13, wherein the one or more antibiotic is selected from the group consisting of a carbapenem, penicillin, a cephalosporin, a glycopeptide, a lipopeptide, a monobactam, an oxazolidinone, a quinolones/fluoroquinolone, a tetracycline, an analog thereof and an antibiotic active against micobacteria.

15- The method of claim 14, wherein the one or more antibiotic is selected from the group consisting of Enrofloxacin, Imipenem/cilastatin, Meropenem, Piperacillin/tazobactam, Ampicillin/sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin and Tigecycline.

16- A pharmaceutical composition comprising CM from MenSCS and one or more antibiotic is selected from the group consisting of a carbapenem, penicillin, a cephalosporin, a glycopeptide, a lipopeptide, a monobactam, an oxazolidinone, a quinolone/fluoroquinolone, a tetracycline, an analog thereof and an antibiotic active against micobacteria.

17- The pharmaceutical composition of claim 16, wherein the one or more antibiotic is selected from the group consisting of Enrofloxacin, Imipenem/cilastatin, Meropenem, Piperacillin/tazobactam, Ampicillin/sulbactam, Clindamycin, Metronidazole, Cefepime, Levofloxacin, Vancomycin, Trimethoprim/sulfamethoxazole, Aztreonam, Linezolid, Ceftriaxone, Daptomycin, Nafcillin, Rifampin, Daptomycin and Tigecycline.

18- A kit comprising at least two components, recipients or vials A and B, wherein the component, recipient or vial A comprises CM of MenSCs and wherein the component, recipient or vial B comprises an antibiotic selected from the group consisting of a carbapenem, penicillin, a cephalosporin, a glycopeptide, a lipopeptide, a monobactam, an oxazolidinone, a quinolone/fluoroquinolone, a tetracycline, an analog thereof and an antibiotic active against micobacteria.
